SEQUENCE LISTING (1 sequence)

SEQ ID NO: 1 - DNA, 18 bp, Artificial Sequence
  Nucleic acid probe
tggaagattccctactgc

(19) United States
(12) Patent Application Publication
(10) Pub. No.: US 2005/0136446 A1
(43) Pub. Date: Jun. 23, 2005

(54) Method for the identification of microorganisms by means of in situ hybridization and flow cytometry

(76) Inventors: Jiri Snaidr, Munich (DE); Claudia Beimfohr, Munich (DE); Karin Thelen, Grafelfing (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/954,077

(22) Filed: Sep. 28, 2004

Related U.S. Application Data
(63) Continuation of application No. PCT/EP03/03204, filed on Mar. 27, 2003.

(30) Foreign Application Priority Data
Mar. 28, 2002 (DE) 102 14 153.3

(51) Int. Cl. C12Q 1/68

(52) U.S. Cl. 435/6

(57) ABSTRACT

The invention relates to a combined method for specifically identifying microorganisms by means of in situ hybridization and flow cytometry. The inventive method is particularly characterized by an improved specificity and a shorter duration of the process as opposed to methods known in prior art.

### CROSS-REFERENCE TO RELATED APPLICATIONS

This application is a continuation of and claims priority under 35 U.S.C. § 120 to PCT Application No. PCT/EP03/03204, filed on Mar. 27, 2003, entitled METHOD FOR THE IDENTIFICATION OF MICROORGANISMS BY MEANS OF IN SITU HYBRIDIZATION AND FLOW CYTOMETRY, which claims priority from German Application No. 102 14 153.3, filed on March 28, 2002; the disclosure of each of which is hereby incorporated by reference in its entirety.

### BACKGROUND OF THE INVENTION

1. Field of the Invention

The invention relates to a combined method for the specific detection of microorganisms by in situ hybridization and flow cytometry. The inventive method is particularly characterized by an improved specificity and a shorter process time as opposed to methods known in the prior art.

2. Description of the Related Art

Traditionally, microorganisms are detected by cultivation. However, this detection method has a number of disadvantages. Particularly in the analysis of the biocoenosis of environmental samples the cultivation has been shown to be completely unsuitable. Cultivation-dependent methods provide only a very false view of the composition and dynamics of the microbial biocoenosis. For example, it could be shown that in recording the flora of the activated sludge by cultivation a cultivation shift occurs (Wagner, M., R. Amann, H. Lemmer and K. H. Schleifer, 1993, Probing activated sludge with oligonucleotides specific for proteobacteria: inadequacy of culture-dependent methods of describing microbial community structure, Appl. Environ. Microbiol. 59:1520-1525).

Because of this medium-dependent distortion of the real conditions within the bacterial population, the importance of bacteria which play only a minor role in activated sludge, but which are well adjusted to the cultivation conditions used, is dramatically overestimated. It could thus be shown that due to such cultivation artifacts the bacterial genus Acinetobacter was completely misjudged with respect to its role as biological phosphate remover in the purification of sewage. Such misconceptions result in the cost-intensive, error-prone and imprecise creation of plants. The efficiency and reproducibility of such simulation calculations is low.

But the cultivation has significant disadvantages also in the analysis of foodstuffs or medical samples. The methods used here are often very tedious, require a multiplicity of successive cultivation steps and produce results which are not infrequently unclear. The testing of a water sample for the presence or absence of faecal streptococci is described here by way of example. The detection methods recommended in the Drinking Water Ordinance are based on the direct cultivation of the water sample or a membrane filtration and subsequent introduction of the filter in 50 ml azide-glucose-broth. The cultivation should be carried out for at least 24 hours, in the case of a negative result for 48 hours, at 36° C. If after 48 hours clouding or sedimentation of the broth is still not detectable, the absence of faecal streptococci in the tested sample is deemed to have been proven. In the case of clouding or sedimentation, streaking of the culture on enterococci selective agar according to Slanetz-Barthley and re-incubation at 36° C. for 24 hours takes place. If reddish-brown or pink colonies form, these will be examined in more detail. After transfer to a suitable liquid medium and cultivation for 24 hours at 36° C., faecal streptococci are deemed to have been detected when propagation in nutrient broth at a pH of 9.6 takes place and the propagation in 6.5% NaCl-broth is possible as well as in the case of esculin degradation. Esculin degradation is checked by the addition of freshly prepared 7% aqueous solution of iron(II) chloride to esculin broth. In the case of degradation a brownish-black color develops. Frequently, a Gram stain for differentiating bacteria from Gram-negative cocci is additionally carried out as well as a catalase test for differentiating from staphylococci. Faecal streptococci react Gram-positive and catalase-negative. The traditional detection procedure is thus shown to be a tedious (48-100 hours) and, in suspected cases, an extremely elaborate method.

Due to the disadvantages of the cultivation described, modem methods for the identification of bacteria all have a common aim: they attempt to get around the disadvantages of cultivation in that they no longer require the cultivation of the bacteria, or at least reduce the cultivation to a minimum.

In PCR, polymerase chain reaction, a characteristic piece of the respective bacterial genome is amplified with primers specific for bacteria. If the primer finds its target site, a million-fold amplification of a piece of the inherited material occurs. Upon the following analysis by an agarose gel separating DNA fragments, a qualitative evaluation can take place. In the simplest case this leads to the conclusion that the target sites are present in the tested sample. Further conclusions are not possible, because the target sites can originate from a living bacterium, a dead bacterium or from naked DNA. Differentiation is not possible with this method. A further refinement of this technique is the quantitative PCR, which tries to establish a correlation between the amount of bacteria present and the amount of DNA obtained and amplified. However, various substances contained in the analyzed sample can lead to an inhibition of the DNA amplifying enzyme, the Taq polymerase. This a common cause of false negative results of the PCR. Advantages of PCR are its high specificity, its ease of application and its low expenditure of time. Its main disadvantages are its high susceptibility to contamination and therefore false positive results, as well as the aforementioned lack of possibility to discriminate between living and dead cells or naked DNA, respectively, and finally the danger of false positive results due to the presence of inhibitory substances.

Also, biochemical parameters are used for the identification of bacteria. Thus, the establishment of bacterial profiles on the basis of quinone determinations serves to render an image of the bacterial population which is as distortion-free as possible (Hiraishi, A. 1988. Respiratory quinone profiles as tools for identifying different bacterial populations in activated sludge. J. Gen. Appl. Microbiol. 34:39-56). This method also is dependent on the cultivation of individual bacteria, since the establishment of the reference database requires the quinone profiles of the bacteria in pure culture. Moreover, the determination of the quinone profiles of the bacteria cannot give a real impression of the actual populations present in the sample.

In contrast hereto, the detection of bacteria by antibodies is a more direct method (Brigmon, R. L., G. Bitton, S. G. Zam, and B. O'Brien. 1995. Development and application of a monoclonal antibody against Thiothrix spp. Appl. Environ. Microbiol. 61: 13-20). Fluorescence labeled antibodies are mixed with the sample and allow a highly specific attachment to the bacterial antigens. The thus labeled bacteria are then detected in the epifluorescence microscope based on their emitted fluorescence. In this way, bacteria can be identified up to the level of the strain. However, there are crucial disadvantages which drastically limit the applicability of this method. First of all, pure cultures of the bacteria to be detected are required for the production of the antibodies. This means of course that ultimately only those bacteria which are cultivatable at all are detectable by antibodies. However, the majority of bacteria is not cultivatable, and can therefore, not be detected using this method. Secondly, the often large and bulky antibody-fluorescence-molecule-complex has problems in entering the target cells. Thirdly, the application of antibodies is limited to certain samples which are present in a suitable form or appropriately prepared. Especially environmental samples, which often have a high percentage of particles (e.g., soil samples or sludge samples), can only be inadequately analyzed by antibodies. In these samples, unspecific adsorption of the antibodies to the particles contained increasingly occurs. This can lead to false positive results, when the fluorescent particles are confused with the bacteria to be detected. The evaluation of the analysis is at least made very difficult, since non-specifically glowing particles have to be distinguished from specifically glowing bacteria. Fourthly, the detection using antibodies is often too specific. The antibodies often detect only a certain bacterial strain of a bacterial species with high specificity, but leave other strains of the same bacterial species undetected. However, in most cases strain-specific detection of bacteria is not required, but rather the detection of all bacteria of a bacterial species or an entire bacterial group. For many bacterial species this has so far not been successful, namely the development of a detection method based on antibodies which detects not only individual strains but all bacteria of a species. Fifthly, the production of antibodies is a relatively tedious and expensive process.

As a novel approach, the method of in situ hybridization with fluorescence labeled oligonucleotide probes was developed at the beginning of the nineties, which is known as fluorescence in situ hybridization (FISH; Amann et al. (1990) J. Bacteriol. 172:762; Amann, R. I., W. Ludwig, and K. -H. Schleifer. 1995. Phylogenetic identification and in situ detection of individual microbial cells without cultivation. Microbiol. Rev. 59:143-169). Using this method, bacterial species, genera or groups may be identified and if necessary, also visualized or quantified directly in a sample with high specificity. This method is the only one providing a distortion-free representation of the actual in situ conditions of the biocoenosis. Even bacteria not cultivated up to now and thus not yet described can be identified.

The FISH technique is based on the fact that in bacterial cells there are certain molecules which have only been mutated to a small extent in the course of evolution because of their essential function. These are the 16S and the 23S ribosomal ribonucleic acid (rRNA). Both are parts of the ribosomes, the sites of protein biosynthesis, and can serve as specific markers on account of their ubiquitous distribution, their size and their structural and functional constancy (Woese, C. R., 1987. Bacterial evolution. Microbiol. Rev. 51, p. 221-271).

For the application of the FISH, so-called gene probes (usually small, 16-25 bases long, single-stranded desoxyribonucleic acid pieces) are developed which are complementary to a defined region of the rRNA. This defined region is selected in such a way that it is specific for a bacterial species, genus or group.

In FISH, labeled gene probes enter the cells present in the tested sample. If a bacterium of the species, genus or group for which the gene probes were developed is present in the sample tested, the gene probe binds to its target sequence in the bacterial cell and the cells can be detected thanks to the labeling of the gene probes.

The advantages of the FISH technique compared to the above described methods for the identification of bacteria (cultivation, PCR or antibodies) are many.

Firstly, using gene probes numerous bacteria can be detected which are not detectable using traditional cultivation. Whereas using cultivation, a maximum of only 15% of the bacterial population of a sample can be visualized, the FISH technique allows detection of up to 100% of the total bacterial population in many samples. Secondly, detection of bacteria using the FISH technique is much faster than using cultivation. Whereas the identification of bacteria by cultivation often takes several days, using the FISH technique there is only a few hours between sampling and the bacteria identification, even on the species level. Thirdly, in contrast to a cultivation medium the specificity of the gene probes can be almost freely selected. Individual species can be detected with one probe just as well as entire genera or bacterial groups. Fourthly, bacterial species or entire bacterial populations can be exactly quantified directly in the sample.

In contrast to PCR, FISH can reliably detect only living bacteria. False positive results by naked DNA or dead bacteria as in the case of PCR are ruled out using FISH. Furthermore, false negative results due to the presence of inhibitory substances are equally ruled out as are false positive results due to contaminations.

In contrast to the antibody technology, the production of nucleic acid probe molecules is simple, fast and inexpensive. Further, the complex of nucleic acid probe molecule and fluorescence stain is by far smaller than the antibody-stain-complex and, in contrast to the latter, can easily enter the cells to be detected. As already described above, also the specificity of the nucleic acid probe molecules can be almost freely selected. Individual species can be detected with a probe as can entire genera or bacterial groups. Finally, in contrast to the antibody technology, the FISH technique is suitable for the testing of many different types of samples.

The FISH technique is thus an outstanding tool for detecting bacteria fast and highly specifically directly in a sample. In contrast to the cultivation method, it is a direct method and moreover also allows a quantification.

Routine FISH analysis is performed on a suitable solid, optically transparent substrate, as e.g., a slide or a micro titer plate. Evaluation is then performed in a microscope, the bacteria being visualized by irradiation with a high-energy light.

The conventional FISH method for the detection of microorganisms using a solid substrate has, however, also its limitations. It cannot be automated, or at least only with difficulty, and is thus comparatively protracted and moreover not always reproducible with the same quality. Furthermore, another possible source of error for quantitative analysis in a microscope is the subjectivity of the observer, which can never entirely be eliminated.

Above all, the lack of automation and the thus comparatively laborious and protracted handling as well as the quantification which is subject to the subjective impression of the observer have led to the fact that the FISH analysis has up to now only been used in industry for single and multiple analysis, but not for high-throughput analysis, and is only rarely used for exact quantification. However, since the analysis of bacteria using this method still has important advantages compared to all other microbiological analysis methods presently used in industry, as described, there is a need for a combined method which combines the advantages of the FISH analysis with those of a fast, simple and objective method which can be automated and which therefore allows the simple, fast and reliable analysis of samples for the identification and quantification of microorganisms.

In the past few years flow cytometry has acquired a strong influence as “automated microscopy,” especially in biological and medical research as well as in diagnostics. It offers the advantages of automation, objectivity and high evaluation speed (several thousand cells can be measured per second).

The flow cytometry allows the counting and the analysis of physical and molecular characteristics of particles (such as e.g., cells) in a liquid stream. Flow cytometry allows the documentation of certain characteristics of cells or cell populations on the single cell level.

Flow cytometry consisting principally of a liquid system and an optical system. The basis for a successful test is the hydrodynamic focussing of the sample by the liquid system. Here, the cells contained in the sample are thinned out and arranged with a very high degree of accuracy (deviation 1 μm) linearly on the measuring point. The optical system generally consists of an excitation light source (e.g., diverse lasers or a mercury pressure lamp), various optical mirrors and detectors for the forward scattered light, the sideways scattered light and the fluorescence light. Flow cytometers are already available from several suppliers, such as e.g., the product Microcyte from Optoflow, Norway, or the BD FACSCalibur apparatus from BD Biosciences, Becton, Dickinson and Company. In addition, companies and institutes offer the performance of corresponding flow cytometry analysis and/or use times for flow cytometers.

At the beginning of the analysis the sample is fed into the center of the transport liquid. The separating and centering of the cells is achieved by the coat stream (Mantelstrom), which is generated by a higher flow speed of the transport liquid compared to the sample stream. By means of the liquid system which is under pressure, the single cells are now passed by the excitation light source of the optical system continuously and with constant speed.

The laser light impacting the cells is first scattered in two directions: the scattered light directed “forward” at an angle of 2-15° (FSC for forward scatter) and “sideways” at an angle of 15-90° (SSC for sideways scatter) is a measure of the size and granularity of the cell. In addition, various fluorochromes can be excited for the emission of light quants via diverse lasers (e.g., argon or helium-neon laser) or a mercury pressure lamp with suitable optical filters. The light quants are then detected by suitable sensors. The measured values obtained are visualized in the form of histograms or dot plots on the computer.

The flow cytometry has however hitherto only been used on a very small scale for microbiological tests. First attempts to combine the FISH and the flow cytometry were performed by Wallner (Wallner, G. et al. (1993) Cytometry 14(2):136-143; Wallner G. et al. (1995) Appl. Environ. Microbiol. 61(5):1859-1866; Wallner, G. et al. (1997) Appl. Environ. Microbiol. 63(11):4223-4231).

Disadvantages of the method described in the prior art are again the relatively tedious procedure (hybridization time of 3 hours, centrifugation step between hybridization and washing, washing time of 0.5 hours), the uncertain specificity of the method as a result of this tedious procedure as well as the unsuitability of this method for the detection of gram-positive bacteria.

It is the object of the present invention to overcome the above-described disadvantages of the prior art and to provide a method by which microorganisms can be detected specifically, simply, reproducibly, reliably, fast and objectively.

### SUMMARY OF THE INVENTION

Some embodiments relate to methods for the detection of microorganisms in a sample by in situ hybridization and flow cytometry. The methods can include the steps of:
a) fixing the microorganisms contained in the sample using a fixing agent,
   i) drying the sample containing the microorganisms and thereby removing the fixing agent;
b) a hybridization step comprising incubating the fixed microorganisms with nucleic acid probe molecules contained in a hybridization solution in order to achieve hybridization;
c) a washing step comprising adding a washing solution to the fixed microorganisms incubated with the nucleic acid probe molecules; and
d) detecting the microorganisms with hybridized nucleic acid probe molecules by flow cytometry, wherein the hybridization solution is not removed between the hybridization step b) and the washing step c).

The microorganism can be a yeast, a bacterium, an alga or a fungus. The microorganism can be a gram-positive bacteria. The methods can further include, between the drying step i) and the hybridization step b), the step of ii) lysing the fixed microorganisms. The nucleic acid probe molecules can be covalently linked to a detectable marker and the detectable marker can be selected from the group consisting of fluorescence markers, chemoluminescence markers, radioactive markers, enzymatically active groups, haptenes, and nucleic acids detectable by hybridization, for example. In some aspects the washing step can be performed for less than 30 minutes. In some aspects the washing step is performed for no longer than 20 minutes, preferably no longer than 15 minutes.

### BRIEF DESCRIPTION OF THE DRAWINGS

FIG. 1: Results of some experiments with negative findings. A: Density plot of Staphylococcus aureus cells stained with the probe Lgc-354a; reading: 1.3×10² counts/ml. B: density plot of Escherichia Coli cells stained with the probe Lgc-354a; reading: 1.0×10³ counts/ml. C: density plot of Salmonella cholerasuis cells stained with the probe Lgc-354a; reading: 3.1×10³ counts/ml. D: density plot of Staphylococcus aureus cells stained with probe Lgc-354a; reading 4.5×10³ counts/ml. E: density plot of Escherichia coli cells stained with probe Lgc-354a; reading: 5.2×10³ counts/ml. F: density plot of Salmonella cholerasuis cells stained with probe Lgc-354a; reading: 6.7×10³ counts/ml.

FIG. 2: Results of some experiments with positive findings. G: Density plot of 1 ml Pediococcus damnosus cells hybridized with the probe Lgc-354a; reading: 5,2×10⁵ counts/ml H: Density plot of 2 ml P. damnosus cells hybridized with the probe Lgc-354a; reading: 9,8×10⁵ counts/ml I: Density plot of 1 ml Lactobacillus brevis cells detected with the probe Lgc-354a; reading: 6,16×10⁵ counts/ml J: Density plot of 2 ml L. brevis cells detected with the probe Lgc-354a; reading: 1,27×10⁶ counts/ml K: Density plot of a mixture of 1 ml P. damnosus cells and 1 ml L. brevis cells stained with the probe Lgc-354a; reading: 1,6×10⁶ L: Density plot of 1 ml L. brevis cells detected with the probe Lgc-354a; reading: 6,34×10⁵ counts/ml. G to K: with centrifugation after the washing step; L: without centrifugation after the washing step.

### DETAILED DESCRIPTION OF THE PREFERRED EMBODIMENT

According to the invention the above-mentioned object is solved by the features of the independent claim. Further embodiments will become clear from the features of the dependent claims.

The implementation of the method according to the invention for the specific detection of microorganisms in a sample comprises the following steps:
a) fixing the microorganisms contained in the sample,
b) incubating the fixed microorganisms with nucleic acid probe molecules contained in a hybridization solution in order to achieve hybridization (=hybridization step),
c) adding a washing solution to the fixed microorganisms incubated with the nucleic acid probe molecules (=washing step),
d) detecting the microorganisms with hybridized nucleic acid probe molecules by flow cytometry,
wherein the hybridization solution is not removed between the hybridization step b) and the washing step c).

In a preferred embodiment the method further comprises between the fixing step a) and the hybridization step b) the step i) drying the sample and removing the fixing agent.

In a further preferred embodiment the method according to the invention further comprises between the fixing step a) and the hybridization step b), or between the drying step i) and the hybridization step b) the step ii) lysing the fixed microorganisms.

A particularly preferred embodiment of the method for the specific detection of microorganisms in a sample therefore provides the following steps:
a) fixing the cells contained in the sample,
   i) drying the sample and removing the fixing agent,
   ii) complete lysis of the cells contained in the sample,
b) incubating the fixed and lysed cells with nucleic acid probe molecules in order to achieve hybridization,
c) adding a washing solution,
d) detecting the cells with hybridized nucleic acid probe molecules in the flow cytometer,
wherein between step b) and step c) the hybridization solution containing the nucleic acid probe molecules is not removed.

Optionally, the first step is preceded by a short cultivation for the enrichment of the cells contained in the sample to be tested.

In a further embodiment the method can be performed without centrifugation after the washing step. By dispensing completely with centrifugation the method according to the invention can be performed even faster and more simply.

Within the scope of the present invention “fixing” of the microorganisms is understood to mean a treatment with which the bacterial envelope is made permeable for nucleic acid probes. For fixation, usually ethanol is used. If the cell wall cannot be penetrated by the nucleic acid probes using these techniques, the expert will know a sufficient number of other techniques which lead to the same result. These include, for example, methanol, mixtures of alcohols, a low percentage paraformaldehyde solution or a diluted formaldehyde solution or the like.

Within the scope of the present invention “drying” is understood to mean an evaporation of the sample at elevated temperature, until the fixation solution is completely evaporated.

Within the scope of the present invention, “complete lysis of the cells” is understood to mean an enzymatic treatment of the cells. By this treatment, the cell wall of gram-positive bacteria is made permeable for nucleic acid probe molecules. For this purpose, for example lysozyme in a concentration of 0.1-10 mg/ml H₂O is suitable. Also, other enzymes, such as for instance mutanolysine or proteinase K can be used alone or in combination. Suitable concentrations and solvents are well known to the expert. It goes without saying that the method according to the invention is also suitable for the analysis of gram-negative bacteria; the enzymatic treatment for complete cell lysis is then adapted accordingly, it can then also be completely dispensed with.

Within the scope of the present invention the fixed bacteria are incubated with fluorescence labeled nucleic acid probe molecules for the “hybridization.” These nucleic acid probe molecules, which consist of an oligonucleotide and a marker linked thereto can then penetrate the cell wall and bind to the target sequence corresponding to the nucleic acid probe molecule within the cell. Binding is to be understood as formation of hydrogen bonds between complementary nucleic acid pieces.

The nucleic acid probe molecule here can be complementary to a chromosomal or episomal DNA, but also to an mRNA or rRNA of the microorganism to be detected. It is advantageous to select a nucleic acid probe molecule which is complementary to a region present in copies of more than 1 in the microorganism to be detected. The sequence to be detected is preferably present in 500-100,000 copies per cell, especially preferred 1,000-50,000 copies. For this reason the rRNA is preferably used as target site, since the ribosomes as sites of protein biosynthesis are present many thousand-fold in each active cell.

The nucleic acid probe molecule within the meaning of the invention may be a DNA or RNA probe usually comprising between 12 and 1,000 nucleotides, preferably between 12 and 500, more preferably between 12 and 200 and between 12 and 100, especially preferably between 12 and 50 and between 14 and 40 and between 15 and 30, and most preferably between 16 and 25 nucleotides. The selection of the nucleic acid probe molecules is done according to criteria of whether a suitable complementary sequence is present in the microorganism to be detected. By selecting a defined sequence, a bacterial species, a bacterial genus or an entire bacterial group may be detected. In a probe consisting of 15 nucleotides, the sequences should be 100% complementary. In oligonucleotides of more than 15 nucleotides, one or more mismatches are allowed.

A sequence is suitable if it is on the one hand specific for the microorganism to be detected and on the other hand accessible for the entering nucleic acid probe molecule, i.e., not masked by ribosomal proteins or the secondary structure of the rRNA.

Within the scope of the present invention the nucleic acid probe molecules are used with suitable hybridization solutions. Suitable compositions of this solution are well known to the expert. Such a hybridization solution contains organic solvents, in particular formamide, in a concentration of between 0% and 80% and has a salt concentration (preferably NaCl) between 0.1 mol/l and 1.5 mol/l. Also contained is a detergent (usually SDS) in a concentration of between 0% and 0.2% as well as a buffer substance suitable for the buffering of the solution (e.g., Tris-HCl, Na-citrate, HEPES, PIPES or similar), usually in a concentration of between 0.01 mol/l and 0.1 mol/l. Usually, the hybridization solution has a pH of between 6.0 and 9.0.

The concentration of the nucleic acid probe in the hybridization solution depends on the kind of its label and on the number of target structures. In order to allow rapid and efficient hybridization, the number of nucleic acid probe molecules should exceed the number of target structures by several orders of magnitude. However, it has to be noted that too high levels of fluorescence labelled nucleic acid probe molecules result in increased background fluorescence. The concentration of the nucleic acid probe molecules should therefore be in the range between 0.5 and 500 ng/μl. Within the scope of the method of the present invention the preferred concentration is 1-10 ng for each nucleic acid probe molecule used per μl hybridization solution. The volume of the hybridization solution used should be between 8 μl and 100 ml, in a preferred embodiment of the method of the present invention it is between 10 μl and 1000 ml, especially preferred it is between 20 μl and 200 μl.

It is characteristic for the method according to the invention that the concentration and the volume of the hybridization solution used are adjusted to the volume of the enzyme solution used in the preceding step, if enzymatic lysis takes place. Immediately after mixing the enzyme and the hybridization solution, the chemicals contained in the hybridization solution are present in the concentration required for the specificity of the detection reaction. At the same time, the hybridization solution is composed in such a way that the enzyme reaction for the cell lysis is stopped by the addition of the hybridization solution. In this way the duration of the enzymatic treatment of the tested probe can be controlled very precisely, without a separate working step for removing the enzyme solution being necessary.

The hybridization usually lasts between 10 minutes and 12 hours, preferably the hybridization lasts for about 1.5 hours. The hybridization temperature is preferably between 44° C. and 48° C., especially preferred 46° C., wherein the parameter of the hybridization temperature as well as the concentration of salts and detergents in the hybridization solution may be optimized depending on the nucleic acid probes, especially their lengths and the degree to which they are complementary to the target sequence in the cell to be detected. The expert is familiar with the appropriate calculations.

According to the invention it is further preferred that the nucleic acid probe molecule is covalently linked with a detectable marker. This detectable marker is preferably selected from the group of the following markers:
fluorescence marker,
chemoluminescence marker,
radioactive marker,
enzymatically active group,
haptene,
nucleic acid detectable by hybridization.

The detectable marker is preferably a fluorescence marker.

Within the scope of the present invention “removing” or “displacing” of the non-bound nucleic acid probe molecules is achieved by the addition of a washing solution. That means, in contrast to the prior art, the hybridization solution is not removed prior to the washing step, e.g., by a centrifugation step. Suitable compositions of this solution are well known to the expert. If desired, this washing solution can contain 0.001-0.1% of a detergent such as SDS, as well as Tris-HCl in a concentration of 0.001-0.1 mol/l, wherein the pH of Tris-HCl is in the range of 6.0 to 9.0. The detergent can be included, but is not absolutely necessary. Furthermore, the washing solution usually contains NaCl, the concentration being from 0.003 mol/l to 0.9 mol/l, preferably from 0.01 mol/l to 0.9 mol/l, depending on the required stringency. Also, the washing solution can contain EDTA, wherein the concentration is preferably 0.005 mol/l. Further, the washing solution can also contain preservatives in suitable amounts which are known to the expert.

It is characteristic for the method according to the invention that the concentration and the volume of the washing solution used are adjusted to the volume of the hybridization solution used in the preceding step. Immediately after mixing the hybridization solution and the washing solution, the chemicals contained in the washing solution are present in the concentration required for the specificity of the detection reaction. In contrast to the method according to the invention, in the prior art the hybridization solution is first removed (e.g., by a centrifugation step) and then the washing solution is added. In this process the temperature of the reaction mixture drops to room temperature, resulting in unspecific false positive results of the detection reaction. In contrast, using the method according to the invention ensures that the temperature can be kept constant during the entire hybridization and washing procedure, thus for the first time guaranteeing the specificity of the detection methods.

The superior specificity of the method according to the invention compared to the prior art could be proven by using different probe molecules and different samples, i.e. different microorganisms. The improved specificity is mainly due to the fact that the hybridization solution is not removed between the hybridization step and the washing step, but that the washing solution is added to the cells to be detected and the hybridization solution.

Very good results were achieved when the volume of the hybridization solution was 50-150 μl, especially preferred 80-120 μl, and when the solution was concentrated 1 to 3-fold, especially preferred 1 to 1.5-fold and when the volume of the washing solution was 20-50 μl, especially preferred 30-40 μl and when the washing solution was concentrated 3 to 6-fold, especially preferred 4 to 5-fold.

The non-bound nucleic acid probe molecules are usually “washed off” at a temperature in the range of 44° C. to 52° C., preferably of 44° C. to 50° C. and especially preferred at 46° C. for 10-40 minutes, preferably for 15 minutes.

The specifically hybridized nucleic acid probe molecules are then detected in the respective cells, provided that the nucleic acid probe molecule is detectable, e.g., by linking the nucleic acid probe molecule to a marker by covalent binding. As detectable markers, for example, fluorescent groups, such as for example CY2 (available from Amersham Life Sciences, Inc., Arlington Heights, USA), CY3 (also available from Amersham Life Sciences), CY5 (also obtainable from Amersham Life Sciences), FITC (Molecular Probes Inc., Eugene, USA), FLUOS (available from Roche Diagnostics GmbH, Mannheim, Germany), TRITC (available from Molecular Probes Inc., Eugene, USA), 6-FAM or FLUOS-PRIME are used, which are well known to the person skilled in the art. Also chemical markers, radioactive markers or enzymatic markers, such as horseradish peroxidase, acid phosphatase, alkaline phosphatase, and peroxidase may be used. For each of these enzymes a number of chromogens are known which may be converted instead of the natural substrate and may be transformed to either coloured or fluorescent products. Examples of such chromogens are listed in the following table:

TABLE

| Enzyme | Chromogen |
|---|---|
| 1. Alkaline phosphatase and | 4-methylumbelliferyl phosphate (*), bis(4- |
| acid phosphatase | methylumbelliferyl phosphate, (*) 3-O- |
| | methylfluorescein, flavone-3-diphosphate |
| | triammonium salt (*), p- |
| | nitrophenylphosphate disodium salt |
| 2. Peroxidase | tyramine hydrochloride (*), 3-(p- |
| | hydroxyphenyl)-propionate (*), p- |
| | hydroxyphenethyl alcohol (*), 2,2′- |
| | azino-di-3-ethylbenzothiazoline |
| | sulfonic acid (ABTS), ortho- |
| | phenylendiamine dihydrochloride, |
| | o-dianisidine, 5-aminosalicylic |
| | acid, p-ucresol (*), 3,3′- |
| | dimethyloxy benzidine, |
| | 3-methyl-2-benzothiazoline |
| | hydrazone, tetramethylbenzidine |
| 3. Horseradish peroxidase | H₂O₂ + diammonium benzidine |
| | H₂O₂ + tetramethylbenzidine |
| 4. β-D-galactosidase | o-nitrophenyl-β-D-galactopyranoside, 4- |
| | methylumbelliferyl-β-D-galactoside |
| 5. Glucose oxidase | ABTS, glucose and thiazolyl blue |

*fluorescence

Finally, it is possible to generate the nucleic acid probe molecules in such a way that another nucleic acid sequence suitable for hybridization is present at their 5′ or 3′ ends. This nucleic acid sequence in turn comprises about 15 to 1,000, preferably 15-50 nucleotides. This second nucleic acid region may in turn be detected by a nucleic acid probe molecule, which is detectable by one of the above-mentioned agents.

Another possibility is the coupling of the detectable nucleic acid probe molecules to a haptene which may subsequently be brought into contact with a haptene-recognising antibody. Digoxigenin may be mentioned as an example of such a haptene. Other examples in addition to those mentioned are well known to the expert.

The final detection of the cells labelled as described above takes place in a flow cytometer. The values obtained from this measurement are visualized in the form of histograms or dot plots on the computer and permit reliable statements about the kind and amount of the bacteria contained.

Furthermore, a kit for carrying out the method according to the invention is provided which contains at least one nucleic acid probe molecule for the specific detection of a microorganism, preferably already in the suitable hybridization solution. Preferably, also the suitable washing solution, the fixation solution as well as the solution for the cell lysis and optionally reaction vessels are included.

Important advantages of the method according to the invention are thus the very easy handling as well as speed, reproducibility, reliability and objectivity with which the specific detection of microorganisms in a sample is possible.

A further advantage is that the advantageous method of in-situ hybridization in solution can now for the first time also be performed for gram-positive organisms. Thus, the combined advantages of the FISH and the flow cytometry can for the first time be used for the analysis of gram-positive organisms.

A further advantage is the hybridization time, which, compared to the prior art, is reduced from 3 hours to preferably 1.5 hours.

A further advantage is the specificity of the method. Here it is crucial that the concentration and the volume of the washing solution used is adjusted to the volume of the hybridization solution used in the preceding step. Immediately after mixing the hybridization solution and the washing solution the chemicals contained in the washing solution are present in the concentration required for the specificity of the detection reaction. According to the techniques of prior art, the hybridization solution has first to be removed (e.g., through a centrifugation step) before the washing solution can be added. In this process the temperature of the reaction mixture drops down to room temperature. At this low temperature the nucleic acid probe molecules used in the hybridization reaction bind non-specifically also in those cells which do not contain the exact target sites for the nucleic acid probe molecules but only similar sequences. In the final detection step also these non-target cells, which are labelled due to the unspecific binding of the nucleic acid probe molecules, are detected. A false positive result is the consequence. In contrast, using the method according to the invention ensures that the temperature remains constant during the whole hybridization and washing procedure, as a result of which the specificity of the detection method is for the first time guaranteed.

A further advantage is the washing time, which is reduced compared to the prior art from 30 minutes to preferably 15 minutes.

The microorganism to be detected by the method according to the invention can be a prokaryotic or a eukaryotic microorganism. In most cases it will be desired to detect unicellular microorganisms. These unicellular microorganisms can also be present in larger aggregates, so-called filaments. Relevant microorganisms are especially yeast, algae, bacteria or fungi.

The method according to the invention may be used in various ways.

For example, environmental samples may be tested for the presence of microorganisms. These samples may be collected from air, water or may be taken from the soil.

Another field of application of the method according to the invention is the control of foodstuffs. In preferred embodiments the food samples are obtained from milk or milk products (yogurt, cheese, sweet cheese, butter, and buttermilk), drinking water, beverages (lemonades, beer, and juices), bakery products or meat products.

The method according to the invention may further be used for testing medicinal samples. It is suitable for the analysis of tissue samples, e.g., biopsy material from the lung, tumor tissue or inflamed tissue, from secretions such as sweat, saliva, semen and discharges from the nose, urethra or vagina as well as for urine and stool samples.

A further field of application for the present method is the testing of sewage, e.g., activated sludge, sludge or anaerobic sludge. Apart from this, it is also suitable for the analysis of biofilms in industrial plants, as well as for testing of naturally forming biofilms or biofilms forming in the purification of sewage. Also the testing of pharmaceutical and cosmetic products, e.g., ointments, creams, tinctures, juices, etc. is possible with the method according to the invention.

The following examples are intended to illustrate the invention without limiting it.

### EXAMPLE 1

### Combined Method for the Specific Detection of Microorganisms Taking as an Example the Detection of Lactobacilli Harmful to Beer

The sample to be tested is cultivated for 24-48 hours in a suitable manner. Various suitable methods and cultivation media are well known to the expert. An aliquot of the culture (e.g., 2 ml) is transferred into a suitable reaction vessel and the cells contained are pelleted by centrifugation (4000×g, 5 min, room temperature).

Then a suitable volume (preferably 20 μl) of the fixation solution is added and the open reaction vessel is incubated at ≧37° C. until the fixation solution is completely evaporated.

Then a suitable volume of the enzyme solution (preferably 30-40 μl lysozyme [1 mg/ml H₂O]) is added and the sample is incubated for 7 minutes at room temperature.

Then a suitable volume (preferably 90-120 μl) of 1.33-fold concentrated hybridization solution containing the labelled nucleic acid probe molecules for the specific detection of lactobacilli harmful to beer is added and the sample is incubated (46° C., 1.5 hours).

Then a suitable volume of 5-fold concentrated washing solution (preferably 30-40 μl) is added and the sample is incubated for another 15 minutes at 46° C.

Then the sample is centrifuged (4000×g, 5 min, room temperature). The supernatant is discarded and the pellet is dissolved in a suitable volume of buffered phosphate solution (preferably 100-200 μl).

The sample thus prepared is now analysed on a flow cytometer (e.g., Microcyte, Optoflow, Norway).

### EXAMPLE 2

### Combined Method for the Specific Detection of Microorganisms Taking as Example the Detection of Lactobacilli

1. Material

1.1 Microorganisms used

| Organism | Name of the strain | | Cultivation conditions |
|---|---|---|---|
| Lactobacillus brevis | WSB | L32 | M11/30° C./ |
| | | | standing - |
| | | | micro-aerophilic |
| Escherichia coli | DSM | 30083 | M1/37° C./ |
| | | | agitated 100 rpm- |
| | | | aerobic |
| Pediococcus damnosus | TUM | 618 | M231/30° C./ |
| | | | standing - |
| | | | micro-aerophilic |
| Salmonella cholerasuis | DSM | 554 | M1/37° C./ |
| | | | agitated 100 rpm - |
| | | | aerobic |
| ssp. cholerasuis | | | |
| Staphylococcus aureus | DSM | 1104 | M1/37° C./ |
| | | | agitated 100 rpm - |
| ssp. aureus | | | aerobic |

The bacteria strains designated DSM are available from the DSMZ (German Collection of Microorganisms and Cell Cultures GmbH, Braunschweig, Germany). The strains WSB L32 and TUM 618 are strains from the laboratory collection of the WSB (Faculty of Technology of Brewery I, Freising-Weihenstephan, Germany) and of the Technical University Munich TUM (Faculty of Microbiology, Freising-Weihenstephan, Germany).

1.2 Media used
Medium 11: MRS MEDIUM

| | | |
|---|---|---|
| Casein-Pepton, tryptic digest | 10.00 | g |
| Meat-Extract | 10.00 | g |
| Yeast-Extract | 5.00 | g |
| Glucose | 20.00 | g |
| Tween 80 | 1.00 | g |
| K₂HPO₄ | 2.00 | g |
| Na-Acetate | 5.00 | g |
| (NH₄)₂ Citrate | 2.00 | g |
| MgSO₄ × 7 H₂O | 0.20 | g |
| MnSO₄ × H₂O | 0.05 | g |
| distilled water | ad 1000.00 | ml |

Adjust the pH to 6.2-6.5.

Medium 231: Pediococcus Damnosus Medium

Adjust the pH of Medium 11 (MRS-Medium) to pH 5.2.

Medium 1: NUTRIENT Medium

| | | |
|---|---|---|
| Peptone | 5.0 | g |
| Meat-Extract | 3.0 | g |
| Distilled water | ad 1000.0 | ml |

Adjust the pH to 7.0.

All aforementioned media used for the cultivation of bacteria are commercially available from the DSMZ (German Collection of Microorganisms and Cell Cultures GmbH, Braunschweig, Germany).

1.3 Solutions Used

Hybridization solution (1.5-fold concentrated)

| | | Final | Final |
|---|---|---|---|
| | | Concentration | Concentration |
| Ingredient | Amount | 1.5-fold | 1-fold |
| NaCl solution (5 mol/l) | 2.7 ml | 1350 mmol/l | 900 mmol/l |
| Tris-HCl Buffer (1 mol/l) | 300 μl | 30 mmol/l | 20 mmol/l |
| Water | 1.7 ml | — | — |
| SDS Solution (10%) | 7.5 μl | 0.015% | 0.01% |
| Formamide | 5.3 ml | 52.5% | 35% |
| Final volume: | 10 ml | — | — |

Washing solution (4-fold concentrated)

| | | Final | Final |
|---|---|---|---|
| | | Concentration | Concentration |
| Ingredient | Amount | 4-fold | 1-fold |
| Tris Buffer (1 Mol/l) | 4 ml | 80 mmol/l | 20 mmol/l |
| NaCl solution (5 mol/l) | 2.8 ml | 280 mmol/l | 70 mmol/l |
| EDTA solution (0.5 Mol/l) | 2 ml | 20 mmol/l | 5 mmol/l |
| Water | 41.0 ml | — | — |
| Final volume: | 50.0 ml | — | — |

2. Implementation

The enrichment of the bacterial cultures to be tested was carried out as described under item “1.1 Microorganisms used”. Then an aliquot of the culture (1-2 ml) was transferred to a reaction vessel and the cells contained were pelleted by centrifugation (4000×g, 5 min, room temperature).

The supernatant was discarded and 15 μl of the fixation solution (99.8% EtOH) were added to the cell pellet and the open reaction vessel was incubated at 46° C. until the fixation solution was completely evaporated.

Then 40 μl of the enzyme solution (Lysozyme [1 mg/ml H₂O]) were added and the sample was incubated for 7 minutes at room temperature.

Then 80 μl 1.5-fold concentrated hybridization solution containing a Cy5-labelled nucleic acid probe molecule (Lgc-354a 5′-TGGAAGATTCCCTACTGC-3′; SEQ ID NO: 1) was added and the sample was incubated (46° C., 1.5 hours).

Then 40 μl 4-fold concentrated washing solution was added and the sample was incubated for a further 15 minutes at 46° C.

Then the sample was centrifuged (4000×g, 5 min, room temperature). The supernatant was discarded and the pellet was dissolved in a suitable volume of buffered phosphate solution (preferably 100-200 μl). This last centrifugation step is optional; alternatively, the sample can also be measured without any centrifugation step directly after the washing step.

The sample prepared in this way was analyzed on a flow cytometer (Microcyte, Optoflow, Norway) using the MC2200 software (Optoflow, Norway). Further, the software WinMDI 2.8 (Windows Multiple Document Interface for Flow Cytometry), a program freely available under http://facs.scripps.edu/software.html, was used for the graphic post-editing of the readings.

Alternative:

Alternatively, the supernatant was discarded after centrifuging the sample aliquot and 5 μl of the enzyme solution (Lysozyme [1 mg/ml H₂O]) was added to the cell pellet and the sample was incubated for 7 minutes at room temperature.

Then 10 μl of the fixation solution (99.8% EtOH) was added and the open reaction vessel was incubated at 46° C. until the fixation solution was completely evaporated.

In this case, the subsequent hybridization step was performed by adding 120 μl 1-fold concentrated hybridization solution (instead of 80 μl 1.5-fold concentrated solution). All other steps remained unchanged.

3. Results

In contrast to the visual inspection on a microscope, the possibility of distinguishing between unspecific binding or artefacts and a specific signal is very limited on the flow cytometer, if these events occur in a similar size range.

It is therefore essential to set a threshold or a detection limit. Readings below this limit are interpreted as background; readings above this limit are evaluated as a positive result.

This detection limit was determined by measuring pure water, 1×PBS, cells hybridized without probe and cells hybridized with a non-binding oligonucleotide probe and was at 9×10³ counts/ml.

3.1 Negative Findings

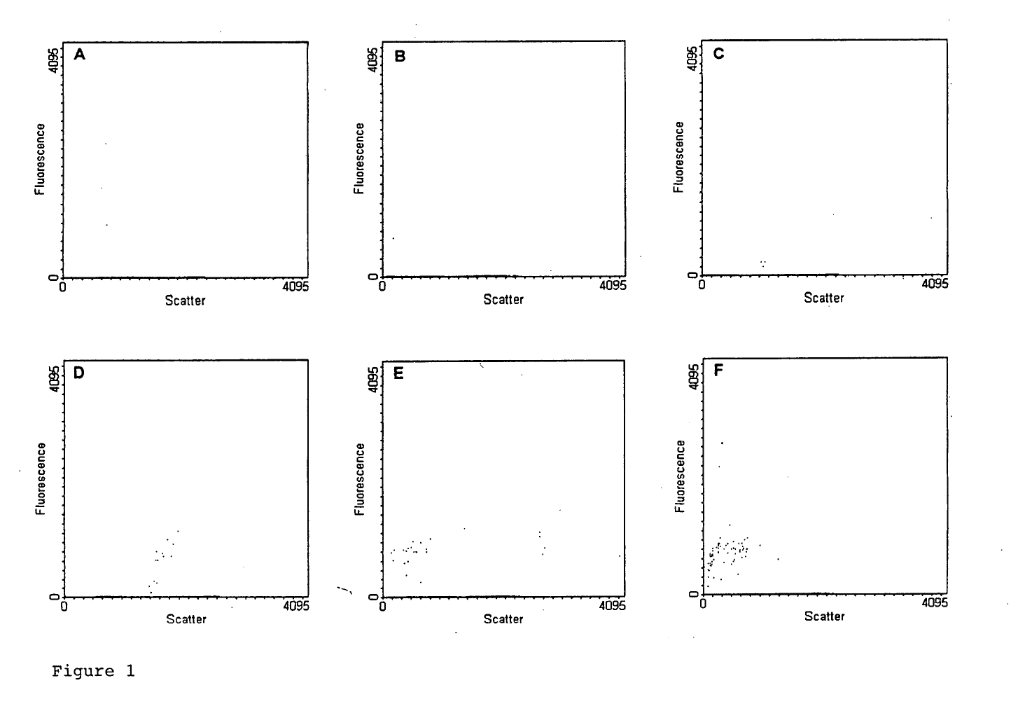

FIG. 1 shows the results obtained with non-target organisms of the probe used. The values obtained were between 1.0×10³ and 3.1×10³ counts/ml (with a centrifugation step after washing, FIG. 1A to C) or between 4.5×10³ and 6.7×10³ counts/ml (without a centrifugation step after washing, FIG. 1D to F), respectively, and were thus clearly below the detection limit. The values were lower with the final centrifugation step than without this step, but also without the final centrifugation step the analysis could be successfully performed.

3.2 Positive Findings

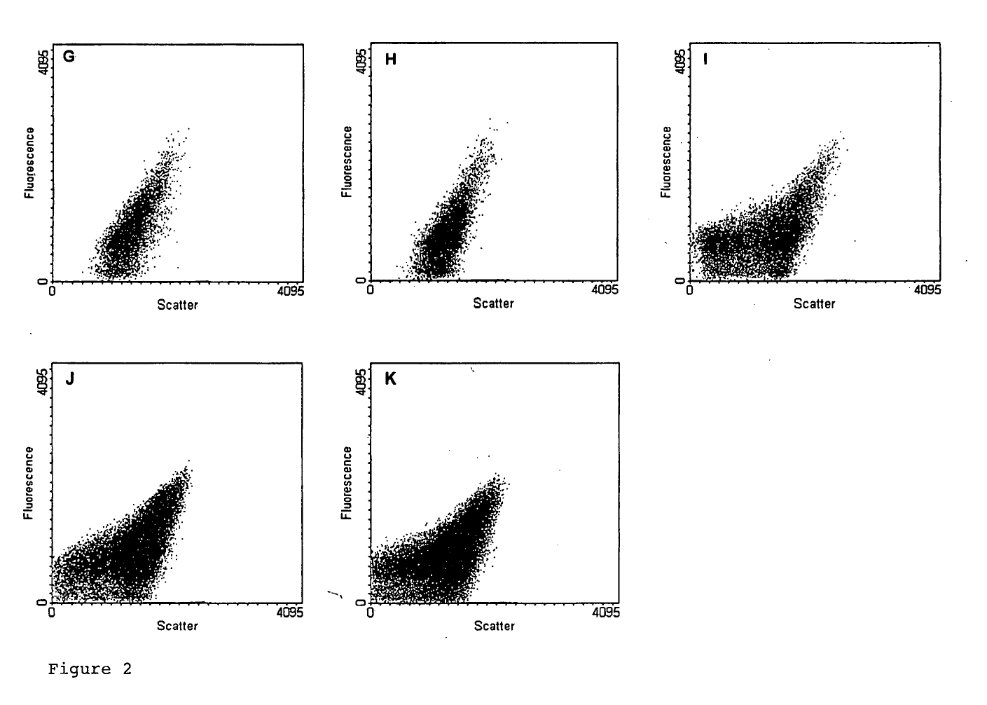

FIG. 2 shows the results obtained with target organisms of the probe used. The values obtained were all clearly above the detection limit. The readings obtained with the analysis of pure and mixed cultures (FIG. 2G-L) were stable and comparable with each other. Also the readings for different amounts of cells (processing of 1 ml or 2 ml of a culture, respectively) showed a good correlation both for Lactobacillus brevis as well as for Pediococcus damnosus.

The measurement of Lactobacillus brevis (see FIG. 2I, J and L) and Pediococcus damnosus (see FIG. 2G and H) cells produced not only reproducible readings, but also different distributions of the single events depending on the morphology of the cells.

The different shape of the plots obtained can primarily be explained by the different morphology (P. damnosus=cocci and L. brevis=rods). Additionally, the homogeneity or the heterogeneity of a culture, respectively, is made clear in the different way of presentation. In this way the culture of P. damnosus consisting of cells of essentially the same size and the same shape is presented conically (see FIG. 2G and H). The distribution of the single readings of the very heterogeneous culture of L. brevis consisting of cells with very different morphology and size (short, long, rods with partially filamentous structures) is presented in the shape similar to a triangle (see FIG. 2I, J and L). The distribution of the single measuring events of a mixed culture of L. brevis and P. damnosus shown in FIG. K shows both different distribution forms in one reading.

1. A method for the detection of microorganisms in a sample by in situ hybridization and flow cytometry, comprising the steps:
a) fixing the microorganisms contained in the sample using a fixing agent,
i) drying the sample containing the microorganisms and thereby removing the fixing agent;
b) a hybridization step comprising incubating the fixed microorganisms with nucleic acid probe molecules contained in a hybridization solution in order to achieve hybridization;
c) a washing step comprising adding a washing solution to the fixed microorganisms incubated with the nucleic acid probe molecules; and
d) detecting the microorganisms with hybridized nucleic acid probe molecules by flow cytometry, wherein the hybridization solution is not removed between the hybridization step b) and the washing step c).

2. The method according to claim 1, wherein the microorganism is a yeast, a bacterium, an alga or a fungus.

3. The method according to claim 1, further comprising between the drying step
i) and the hybridization step b), the step:
ii) lysing the fixed microorganisms.

4. The method according to claim 3, wherein the microorganism is a yeast, a bacterium, an alga or a fungus.

5. The method according to claim 3, wherein the microorganisms are gram-positive bacteria.

6. The method according to claim 1, wherein the nucleic acid probe molecules are covalently linked to a detectable marker and wherein the detectable marker is selected from the group consisting of fluorescence markers, chemoluminescence markers, radioactive markers, enzymatically active groups, haptenes, and nucleic acids detectable by hybridization.

7. The method according to claim 3, wherein the nucleic acid probe molecules are covalently linked to a detectable marker and wherein the detectable marker is selected from the group consisting of fluorescence markers, chemoluminescence markers, radioactive markers, enzymatically active groups, haptenes, and nucleic acids detectable by hybridization.

8. The method according to claim 5, wherein the nucleic acid probe molecules are covalently linked to a detectable marker and wherein the detectable marker is selected from the group consisting of fluorescence markers, chemoluminescence markers, radioactive markers, enzymatically active groups, haptenes, and nucleic acids detectable by hybridization.

9. The method according to claim 1, wherein the washing step is performed for less than 30 minutes.

10. The method according to claim 8, wherein the washing step is performed for no longer than 20 minutes, preferably no longer than 15 minutes.

11. The method according to claim 3, wherein the washing step is performed for less than 30 minutes.

12. The method according to claim 10, wherein the washing step is performed for no longer than 20 minutes, preferably no longer than 15 minutes.

13. The method according to claim 5, wherein the washing step is performed for less than 30 minutes.

14. The method according to claim 13, wherein the washing step is performed for no longer than 20 minutes, preferably no longer than 15 minutes.
